(19) United States
(12) Patent Application Publication
(10) Pub. No.: US 2024/0189563 A1
(43) Pub. Date: Jun. 13, 2024

(54) Medicated therapeutic brace

(71) Applicant: Edwin E. Spencer, Jr., Knoxville, TN (US)

(72) Inventor: Edwin E. Spencer, Jr., Knoxville, TN (US)

(21) Appl. No.: 18/534,072

(22) Filed: Dec. 8, 2023

Related U.S. Application Data
(60) Provisional application No. 63/431,187, filed on Dec. 8, 2022.

(51) Int. Cl. A61M 35/00; A61F 5/01

(52) U.S. Cl. CPC A61M 35/10; A61F 5/0109; A61F 5/0118

(57) ABSTRACT

Apparatus for medication delivery to a patient wearing a therapeutic brace. The medication delivery system includes a medication delivery device working in conjunction with a brace. The delivery device includes a bladder formed from two opposing sheets that are joined at a peripheral edge. The sheet positioned adjacent the brace is impermeable to the medication and the opposite sheet is permeable and is in contact with the epidermis of the patient when the brace is worn. In one embodiment, the device includes a port that extends from the bladder through the brace, allowing medication to be introduced into the bladder when the delivery device is attached to the brace. In another embodiment, the port extends longitudinally from the bladder. A sealing sheet is releasably attached to the bladder to seal the medication in the bladder.

### BACKGROUND

### 1. Field of Invention

This invention pertains to a medicated therapeutic brace. More particularly, this invention pertains to a medication delivery system incorporated into a therapeutic brace whereby a medication is delivered to a patient wearing the brace.

### 2. Description of the Related Art

Various types of medication delivery systems are known. A common medication delivery system is a medicated patch, such as described in European Patent Number 0190262 and related U.S. Pat. No. 5,186,938, both titled “Adhesive transdermal dosage layer.” The patents describe one type of a medicated patch for the sustained release of a pharmaceutically active drug to the skin of a human patient.

U.S. Pat. No. 11,058,803, titled “Medical devices and implements with liquid-impregnated surfaces,” describes one specific way of delivering medication with a brace. The '803 patent describes medical devices and implements with liquid-impregnated surfaces for enhanced lubricity to flesh (or biological fluid) and/or inhibited nucleation on the surface of the device/implement. The surface of the device has an impregnating liquid that is contained between multiple micro-scale and/or nano-scale solid features that are spaced close together. That is, the impregnating liquid fills the spaces between the micro or nano-scaled solid features.

U.S. Published Patent Application Number 2007/0053966, titled “Medicated orthopedic support structures for treatment of damaged musculoskeletal tissue,” very generally describes a orthopedic support device and a nitroglycerin-containing composition. The Abstract contains the statement, “The nitroglycerin-containing composition can be integrated into the orthopedic support structure or the support structure can be configured to contain removable doses of the nitroglycerin-containing composition. The system and method provides relief of pain and improved function of the musculoskeletal tissue.”

The '53966 application generically describes orthopedic support structures as including “braces, compression bandages, structurally supporting tape wraps, elastic sleeves, ceramic, plaster and fiberglass casts, etc.” Publication, para. 40. The application excludes “standard transdermal patches and non-supportive bandages.” Id. When the support structure is a brace, the brace has the medicated composition incorporated in the brace as a removable insert, such as a transdermal patch retained in a pouch on the brace. Para. 40. The “pouch can be partially open so that the nitroglycerin patch can contact the skin freely, or can include a permeable membrane or mesh that allows the drug to adequately contact the skin surface.” Id. In another embodiment, “the nitroglycerin-containing composition can be sewn into the material of the orthopedic support. In [yet] another embodiment the orthopedic support can act as a backing layer onto which the nitroglycerin-containing composition is disposed and adhered.” Para. 41. When the brace is a flexible or elastic sleeve, the “composition can be sewn into the sleeve or can be integrated directly into the sleeve, with the sleeve material acting as a backing layer for the nitroglycerin-containing composition.” Publication, para. 42.

The '53966 application describes that when the support structure is an athletic support tape, the “composition can be incorporated or integrated into the adhesive portion of the tape, or a portion of the tape. The tape can be manufactured by depositing the nitroglycerin-containing adhesive composition onto the tape substrate and allowing the nitroglycerin-containing composition to dry . . . . When tape is used as the orthopedic support structure, the tape substrate can act as a backing layer.” Publication, para. 43. The '53966 application describes a method of preparing the nitroglycerin-containing composition as part of an adhesive matrix. Paras. 45-50. In one embodiment, the resulting nitroglycerin-containing composition is laminated to or coated onto a portion of the orthopedic support structure, with the structure forming a backing layer for the composition. Publication, para. 51.

### BRIEF SUMMARY

A medication delivery system that is a combination of a therapeutic brace and medication delivery device is provided. In various embodiments, the brace is rigid, flexible, or a type that includes both rigid and flexible portions. Examples of common braces include a foot boot, knee brace, back brace, and knee or elbow sleeves. The brace has a surface configured to be positioned proximate to and in contact with the epidermis or skin of the patient wearing the brace. A portion of that surface includes a medication delivery device that dispenses the medication over time as the brace is worn.

In one embodiment, the medication delivery system includes a brace and a releasably attachable medication delivery device. The medication delivery device includes a bladder that contains the medication to be dispensed. In one embodiment, the bladder has a port extending through an opening in the brace. The bladder includes two sheets joined together at their peripheral edge so as to form a sealed container. One sheet is configured to be positioned adjacent the recess in the brace. The brace-sided sheet is an impermeable material with a port. The other, outer sheet is configured to be positioned proximate to and in contact with the epidermis or skin of the patient wearing the brace. The outer sheet is permeable to the medication in the bladder.

The bladder is releasably attachable to the recess in the brace. In one such embodiment, the bladder is secured in the recess by an adhesive. For example, the brace-side sheet has a weak adhesive that holds the bladder in the recess when the brace is not being worn by the patient. In another example, the recess in the brace has a weak adhesive that engages the brace-side sheet of the bladder. With the bladder secured in the recess, the port extends from the brace-side sheet through an opening in the recess. The port is then accessible for refilling the bladder while the brace is being worn by the patient.

Another embodiment of the medication delivery system includes the bladder being incorporated in the structure of the brace. The brace has a resilient layer positioned proximate the patient. The resilient layer includes a small chamber or cavity. The surface of the chamber proximate the resilient layer is impermeable. A permeable sheet or membrane encloses an opposite side of the chamber. The membrane is proximate to and in contact with the epidermis or skin of the patient. An externally accessible port passes through the resilient layer such that the medication can be introduced into the chamber. The chamber or cavity is a reservoir for the medication, which is delivered to the patient through the permeable membrane.

Yet another embodiment of the medication delivery system includes a vesicle-type medication delivery device disposed on the surface of the brace in contact with the patient's skin. In one such embodiment, the medication delivery device includes medication infused in the portion of brace facing the patient. In another embodiment, the medication delivery device includes medication in one or more vesicles on the patient facing surface of the brace. The vesicle version delivers the medication through various mechanisms, including a vesicle with a permeable surface, a vesicle of a specific size, and/or a multitude of small vesicles with a specific life such that the vesicles rupture or dissolve over a period of time that corresponds to the treatment time. In one embodiment of the vesicle-type device is integrated with the brace and in another embodiment the vesicle-type device is configured to fit into a recess in the brace.

### BRIEF DESCRIPTION OF THE SEVERAL VIEWS OF THE DRAWINGS

The above-mentioned features will become more clearly understood from the following detailed description read together with the drawings in which:

FIG. 1 is a cross-sectional view of a first embodiment of a medication delivery system that includes a brace and a medication delivery device.

FIG. 2 is an exploded view of the embodiment of the medication delivery system shown in FIG. 1.

FIG. 3 is a plan view of a first embodiment of the medication delivery device shown in FIG. 1.

FIG. 4 is an exploded view of the medication delivery device shown in FIG. 1.

FIG. 5 is a plan view of another embodiment of the medication delivery device shown in FIG. 1.

FIG. 6 is a cross-sectional view of a second embodiment of a medication delivery device that includes a brace and an integral medication delivery device.

FIG. 7 is an exploded view of the embodiment of the medication delivery device shown in FIG. 6.

FIG. 8 is a cross-sectional view of a third embodiment of a medication delivery system.

FIG. 9 is a plan view of the patient-facing side of the medication delivery system shown in FIG. 8.

### DETAILED DESCRIPTION

Apparatus for a medication delivery system is disclosed. The medication delivery system is generally indicated as 100, with particular embodiments and variations shown in the figures and described below having an alphabetic suffix, for example, 100-A, 100-B, 100-C. Various components are illustrated both generically and specifically in the figures and in the following description. For example, the medication delivery devices 102-A, 102-B, 102-C are discussed individually and separately to ensure clarity when describing the configuration of each medication delivery device 102-A, 102-B, 102-C. The medication delivery device 102, when referred to collectively, is referenced without the alphanumeric suffix.

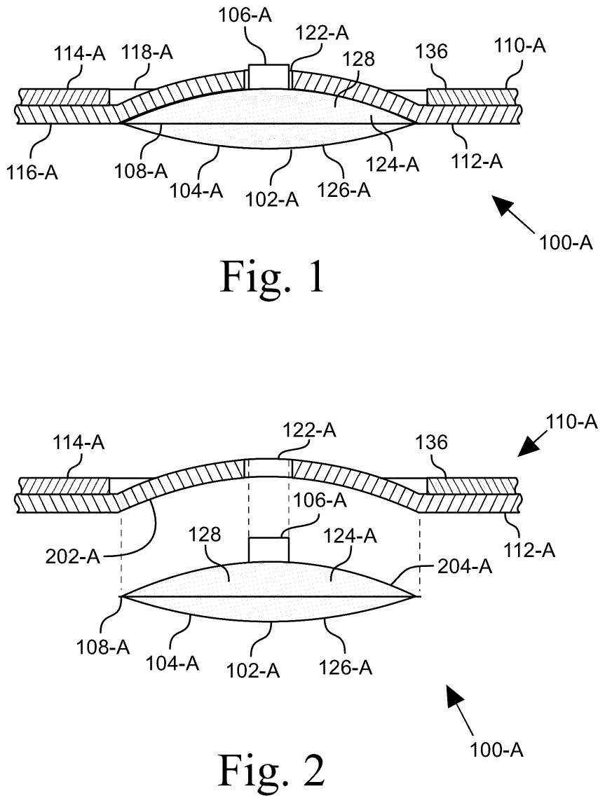

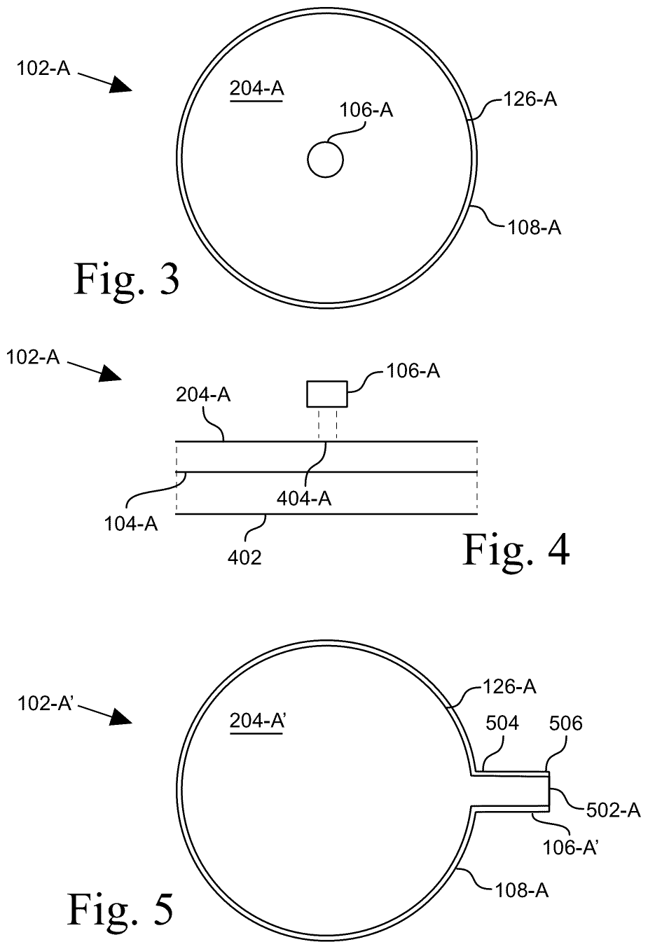

FIG. 1 illustrates a cross-sectional view of a first embodiment of a medication delivery system 100-A that includes a brace 110-A and a medication delivery device 102-A. FIG. 2 illustrates an exploded view of the embodiment of the medication delivery system 100-A that includes the brace 110-A and medication delivery device 102-A shown in FIG. 1. FIG. 3 illustrates a plan view of a first embodiment of the medication delivery device 102-A shown in FIG. 1.

The illustrated brace 110-A includes a rigid portion 114-A and a resilient portion 112-A. In various embodiments, the brace 110 is configured for a specific body part, such as a knee or a foot. In various embodiments, the brace 110 is configured to rigidly conform to the body part or to be articulated, such as to enable movement around a joint, for example, the knee. In such embodiments, the rigid portion 114-A is configured to meet the therapeutic needs of the patient. In another embodiment, the brace 110 is configured to fit flexibly around the body part, for example a sleeve that fits over a body part such as a knee or elbow. In such embodiments, the brace 110 does not include the illustrated rigid portion 114-A.

The resilient portion 112-A is the patient-facing portion of the brace 110-A. In one embodiment, the resilient portion 112-A is flexible layer flexible layer 112-A that is a cushioning material, such as foam or neoprene or other material suitable for being disposed proximate the skin or epidermis of the patient.

In the illustrated embodiment, the brace 110-A includes a recess or depression 202-A on the patient-facing surface 116-A. The rigid portion 114-A has an opening 118-A sized to accommodate the portion of the depression 202-A that protrudes toward the outer side of the brace 110-A. The depression 202-A is circular so that the circular configuration of the medication delivery device 102-A fits in the depression 202-A. The depression 202-A includes an opening 122-A that receives a port 106-A extending from the bladder 126-A. In another embodiment, the resilient portion 112-A is substantially flat with no depression or a shallow depression 202-A. In such an embodiment, the medication delivery device 102-A is attached to the patient-facing surface 116-A and, when the brace 110-A is worn by the patient, the medication delivery device 102-A is squeezed between the resilient layer 112-A and the patient, thereby pushing the resilient layer 112-A into the opening 118-A forming a depression 202-A in the resilient layer 112-A.

The medication delivery device 102-A includes a bladder 126-A and a port 106-A. The bladder 126-A defines a cavity 124-A that receives, contains, and dispenses a medication 128. The port 106-A extends through the brace 110-A, thereby allowing medication 128 to be introduced through the port 106-A from the outer side 136 of the brace 110-A.

The bladder 126-A includes a proximal sheet 204-A and a distal sheet 104-A that are joined at a peripheral edge 108-A. The proximal sheet 204-A and the joined distal sheet 104-A define a cavity 124-A configured to receive, contain, and dispense a medication 128. In one embodiment the proximal sheet 204-A is a membrane that is impermeable to the medication 128 contained in the cavity 124-A. The distal sheet 104-A is a membrane that is permeable to the medication 128 contained in the cavity 124-A. The distal sheet 104-A is configured to allow the medication 128 to pass through the membrane at a desired rate such that the medication 128 is absorbed by the patient's skin, which is in contact with the distal sheet 104-A. The desired rate of medication 128 passing through the distal sheet 104-A is at a rate where the medication 128 has a therapeutic effect on the patient.

The medication delivery device 102-A engages the depression 202-A on the patient-facing surface 116-A of the brace 110-A. In various embodiments, an adhesive is disposed on either one or both of the surface of the depression 202-A and/or the outer surface of the proximal sheet 204-A. In this way, the medication delivery device 102-A is secured to the brace 110-A. In one such embodiment, the adhesive is weak whereby the medication delivery device 102-A is releasable from the brace 110-A after medication delivery device 102-A is secured within the depression 202-A.

When the medication delivery device 102-A engages the depression 202-A the port 106-A engages the opening 122-A in the resilient layer 112-A. In one such embodiment, the opening 122-A is sized to grip the sides of the port 106-A, thereby helping to keep the port 106-A in position when medication 128 is inserted into the medication delivery device 102-A.

FIG. 3 illustrates a plan view of the first embodiment of the medication delivery device 102-A. FIG. 4 illustrates an exploded view of the medication delivery device 102-A shown in FIG. 1. The medication delivery device 102-A includes a bladder 126-A formed of two sheets, namely a proximal sheet 204-A and a distal sheet 104-A. The two sheets 204-A, 104-A and the peripheral edge 108-A joining the sheets 204-A, 104-A define the cavity 124-A. Attached to the outside of the proximal sheet 204-A is a port 106-A. The two sheets 204-A, 104-A are joined together at a peripheral edge 108-A. In one embodiment, the sheets 204-A, 104-A are thermoplastic sheets that are welded together at the peripheral edge 108-A to form a sealed cavity 124-A therebetween.

In one embodiment, the port 106-A is port or inlet that allows medication 128 to be injected into the cavity 124-A of the bladder 126-A. In one such embodiment, the port 106-A is self-sealing after the medication 128 is injected into the cavity 124-A. The port 106-A engages an opening 404-A in the proximal sheet 204-A such that the medication 128 flows from the port 106-A into the cavity 124-A. In one embodiment, the port 106-A and the proximal sheet 204-A are thermoplastic and welded together to form a sealed connection therebetween. In another embodiment, the port 106-A and the proximal sheet 204-A are joined with an adhesive.

A sealing sheet 402 is disposed proximate the distal sheet 104-A until the medication delivery device 102 is deployed for use. The sealing sheet 402 is an impermeable membrane that covers the distal sheet 104-A and prevents the medication 128 from being delivered to the patient. The sealing sheet 402 is weakly affixed to the distal sheet 104-A, such as by a weak adhesive or electrostatic attraction. When delivery of the medication 128 is desired, such was when the brace 110-A is to be worn by the patient, the sealing sheet 402 is peeled from the distal sheet 104-A and discarded, thereby exposing the distal sheet 104-A.

FIG. 5 illustrates a plan view of another embodiment of the medication delivery device 100-A′. In the illustrated embodiment, the port 106-A attached to the proximal sheet 204-A as shown in FIGS. 1-6 is omitted. In its place is a side port or inlet 106-A′ that extends longitudinally from the bladder 126-A. The side port 106-A′ extends longitudinally from the peripheral edge 108-A of the bladder 126-A. The side port 106-A′ includes a neck 504 connecting the side port 106-A′ to the bladder 126-A. The side port 106-A′ includes a distal end 506 opposite the neck 504. The distal end 506 includes opening 502-A, which is one end of a passageway through the port 106-A′ into the bladder 126-A.

The side port 106-A′ allows the medication delivery device 102-A′ to be a single-use device that is filled with medication 128 before being deployed. The medication 128 enters through an opening 502-A in the distal end 506 of the port 106-A′. For example, the distal end 506 of the port 106-A′ receives a tube through which the medication 128 flows through the port 106-A′ and into the cavity 124-A. After the cavity 124-A is filled with medication 128, the port 106-A′ is sealed or welded at the neck 504 where the port 106-A′ joins the cavity 124-A.

In one embodiment, the two sheets 204-A′, 104-A have an extended portion that, when joined together, form a passageway into the cavity 124-A defined between the two sheets 204-A′, 104-A. The sealed peripheral edge 108-A extends around the sheets 204-A′, 104-A and along the sides of the side port 106-A′.

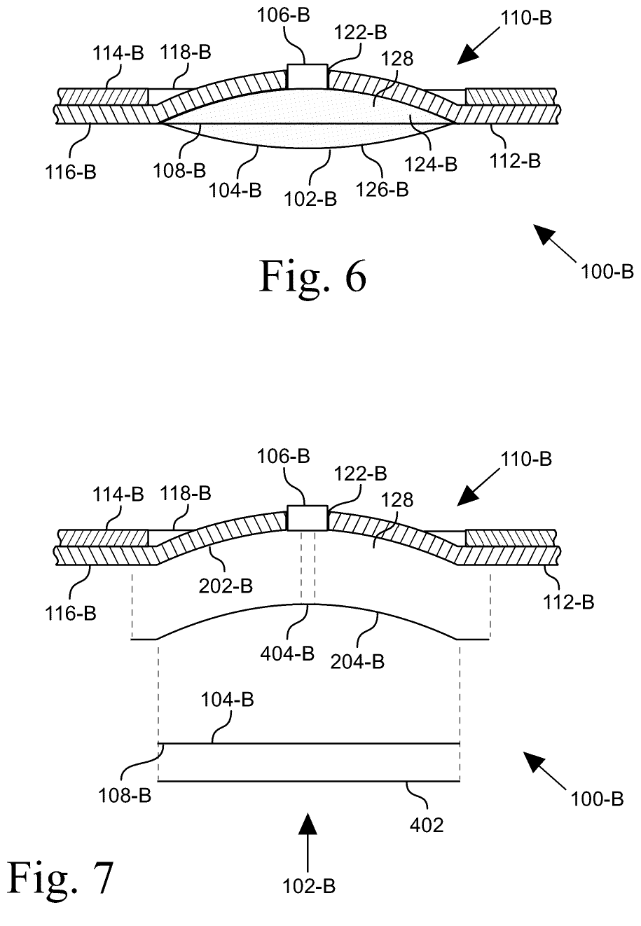

FIG. 6 illustrates a cross-sectional view of a second embodiment of a medication delivery system 100-B that includes a brace 110-B and an integral medication delivery device 102-B. FIG. 7 illustrates an exploded view of the embodiment of the medication delivery system 100-B shown in FIG. 6. In the illustrated embodiment, the medication delivery device 102-B is integrated into the brace 110-B.

The medication delivery device 102-B includes a port 106-B, a proximal sheet 204-B, and a distal sheet 104-B. The proximal sheet 204-B, and the distal sheet 104-B are joined at a peripheral edge 108-B. A cavity 124-B is defined by the space between the proximal sheet 204-B, and the distal sheet 104-B.

The port 106-B engages an opening 122-B in the flexible layer 112-B of the brace 110-B. In one embodiment, the port 106-B is secured to the flexible layer 112-B, such as with an adhesive or by plastic welding. In the illustrated embodiment, the flexible layer 112-B has a depression 202-B that extends through an opening 118-B in the rigid portion 114-B of the brace 110-B. The port 106-B is disposed in the depression 202-B. In another embodiment, the resilient portion 112-B is substantially flat with no or a shallow depression 202-B. In such an embodiment, when the brace 110-B is worn by the patient, the medication delivery device 102-B is squeezed between the resilient layer 112-B and the patient, thereby allowing a portion of the resilient layer 112-B to be pushed into the opening 118-B, thereby forming a depression 202-B in the resilient layer 112-B.

The proximal sheet 204-B is a membrane that is impermeable to the medication 128 contained in the cavity 124-B. The proximal sheet 204-B includes an opening 404-B that is aligned with the port 106-B such that the medication 128 flows from the port 106-B into the cavity 124-B. In one embodiment, the port 106-B and the proximal sheet 204-B are thermoplastic and welded together to form a sealed connection therebetween. In another embodiment, the port 106-B and the proximal sheet 204-B are joined with an adhesive.

The proximal sheet 204-B is attached to the patient-sided surface 116-B of the brace 110-B. In the illustrated embodiment, the proximal sheet 204-B is slightly larger than the distal sheet 104-B. In another embodiment, the entire patient-sided surface 116-B of the brace 110-B is covered by a proximal sheet 204-B. In another embodiment, the entire patient-sided surface 116-B of the brace 110-B is coated with an impermeable material or is otherwise impermeable to the medication 128.

The distal sheet 104-B is a membrane that is permeable to the medication 128 contained in the cavity 124-B. The distal sheet 104-A is configured to allow the medication 128 to pass through the membrane at a desired rate such that the medication 128 is absorbed by the patient's skin, which is in contact with the distal sheet 104-A. The desired rate of medication 128 passing through the distal sheet 104-A is at a rate where the medication 128 has a therapeutic effect on the patient.

The peripheral edge 108-B of the distal sheet 104-B is attached to the proximal sheet 204-B such that the distal sheet 104-B and the proximal sheet 204-B define a cavity 124-B therebetween.

A sealing sheet 402 is disposed proximate the distal sheet 104-B. The sealing sheet 402 is an impermeable membrane that covers the distal sheet 104-B and prevents the medication 128 from being delivered to the patient. The sealing sheet 402 is weakly affixed to the distal sheet 104-B, such as by a weak adhesive or electrostatic attraction. When delivery of the medication 128 is desired, such was when the brace 110-B is to be worn by the patient, the sealing sheet 402 is peeled from the distal sheet 104-B and discarded, thereby exposing the distal sheet 104-B.

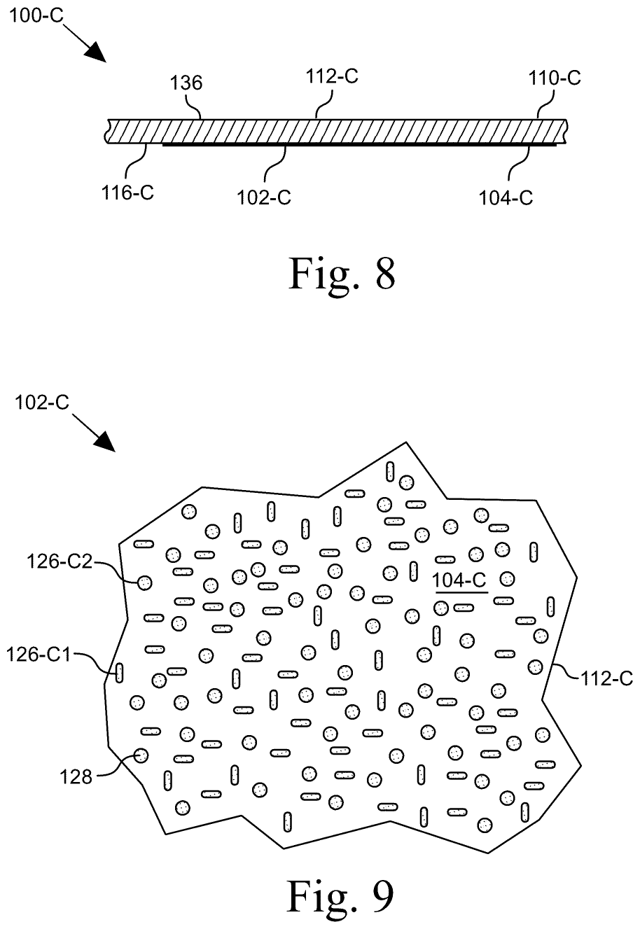

FIG. 8 illustrates a cross-sectional view of a third embodiment of a medication delivery system 100-C. FIG. 9 illustrates a plan view of the patient-facing side of the medication delivery system 100-C shown in FIG. 8. The medication delivery system 100-C is a vesicle-type medication delivery system that includes a brace 110-C and a medication delivery device 102-C disposed on the patient-facing 116-C surface of the brace 110-C. In one embodiment, the brace 110-C is a flexible brace, such as a knee or elbow sleeve and the medication delivery device 102-C is integrated with the brace 110-C.

The illustrated medication delivery device 102-C includes a multitude of cavities 126-C1, 126-C2 that are vesicles that each contain an amount of medication 128. In the illustrated embodiment, the vesicles or cavities 126-C1, 126-C2 have different shapes and are randomly disposed across the area of the medication delivery device 102-C. In other embodiments, the vesicles or cavities 126-C1, 126-C2 have a uniform shape and/or are disposed in a regular pattern across the area of the medication delivery device 102-C.

A distal sheet 104-C is configured to be disposed in contact with the skin or epidermis of the patient. The vesicles or cavities 126-C1, 126-C2 are positioned between the distal sheet 104-C and the flexible layer 112-C. The distal sheet 104-C is permeable to the medication 128 in the vesicles or cavities 126-C1, 126-C2. In one embodiment, the permeability of the distal sheet 104-C various such that the vesicles or cavities 126-C1, 126-C2 dispense medication 128 at different rates and at different times so that the medication 128 is dispensed according to a desired treatment time. In one embodiment, a sealing sheet 402 is disposed proximate the distal sheet 104-C until the medication delivery device 102-C is deployed for use.

In one embodiment, the brace 110-C of the medication delivery system 100-C is a sleeve of an elastic material. In such an embodiment, the medication delivery device 102-C is disposed on an inside or patient-facing surface of the sleeve 110-C.

In another embodiment, the medication delivery device 102-C is sized and configured to fit with the recess 202 such as illustrated in FIGS. 1 & 2. In such an embodiment, the flexible layer 112-C is independent of the brace 110-C, that is, the layer 112-C is sized such that the medication delivery device 102-C fits in the recess 202 with the distal sheet 104-C configured to contact the skin of the person wearing the brace 110-C. In one such embodiment, the medication delivery device 102-C is weakly adhered in the recess 202 such that the medication delivery device 102-C is replaceable.

The medication delivery system 100 includes various functions. The function of dispensing medication 128 to the skin of a patient that is covered by a brace 110 is implemented, in various embodiments, by a medication delivery system 100 that includes a brace 110 and a medication delivery device 102 where the medication delivery device 102 is disposed on the brace 110 proximate the area to be treated by the medication 128.

The function of holding a medication delivery device 102 in position against the skin of a patient that is covered by a brace 110 is implemented, in various embodiments, by a resilient portion or flexible layer 112 of brace. In one embodiment, the medication delivery device 102-A is releasably attachable to the flexible layer 112-A such that the port 106-A extending from the bladder 126-A of the medication delivery device 102-A extends through the flexible layer 112-A. In a second embodiment, the medication delivery device 102-B is integrated with the flexible layer 112-B with the port 106-B extending from the cavity 124-B of the medication delivery device 102-B extends through the flexible layer 112-B. In a third embodiment, the medication delivery device 102-C incorporates a multitude of vesicles or cavities 126-C1, 126-C2 dispense medication 128 as illustrated in FIGS. 8 & 9.

The function of filling a medication delivery device 102 with medication 128 is implemented, in various embodiments, by a port 106-A, 106-B through which the medication 128 can be injected into the cavity 124-A, 124-B of the medication delivery device 102-A, 102-B. In another embodiment, the function of filling the delivery device 102-A′ with medication 128 is implemented by a side port 106-A′ formed from the proximal and distal sheets 204-A′, 104-A. The side port 106-A′ is sealed after the medication 128 is introduced into the cavity 124-A.

The function of sealing the medication delivery device 102 to prevent the delivery of medication 128 until desired is implemented by the sealing sheet 402 that is weakly attached to the distal sheet 104. When delivery of medication 128 is desired, the sealing sheet 402 is peeled away from the distal sheet 104.

From the foregoing description, it will be recognized by those skilled in the art that a medication delivery system 100 that includes a brace 110 and a medication delivery device 102 has been provided. In various embodiments, the medication delivery device 102 includes a bladder 126 that engages a recess 202 on an inside surface of a brace 110. In other embodiments, the medication delivery device 102 includes a vesicle-type medication delivery device 102-C that is either incorporated in a brace 110-C or is sized and configured to engage a recess 202 in the brace 110.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. A medication delivery apparatus for dispensing a medication to a body part of a patient over a period of time, said apparatus comprising:
a brace configured to attach to the patient, said brace having a patient-facing side, said brace having a recess in said patient-facing side, and
a medication delivery device having a bladder and a port, said medication delivery device engaging said recess in said brace, said bladder having a first sheet and a second sheet, said first sheet being a first membrane that is impermeable to the medication, said second sheet being a second membrane that is permeable to the medication, and said first sheet attached to said patient-facing side of said brace.

2. The apparatus of claim 1 wherein said brace includes a flexible portion that has a surface that faces the patient when said brace is attached to the patient, said first sheet releasably attached to said flexible portion, said port passing through an opening in said flexible portion, said port attached to said first sheet with a sealed connection, and said port configured to receive the medication for introduction into a cavity defined by said first sheet and said second sheet.

3. The apparatus of claim 2 wherein said first sheet attached to said flexible portion with a weak adhesive such that said medication delivery device is removable from said brace.

4. The apparatus of claim 2 wherein said port extends through an opening in said brace whereby said medication delivery device is able to receive the medication from an outer side of said brace.

5. The apparatus of claim 2 wherein said port extends longitudinally from a peripheral edge of said medication delivery device, and said port configured to receive the medication for introduction into said cavity.

6. The apparatus of claim 2 wherein said port extends longitudinally from a peripheral edge of said medication delivery device, and said port includes a neck device with a distal end, said distal end includes an opening configured to receive the medication for introduction into said cavity.

7. The apparatus of claim 1 wherein said medication delivery device is integrated with said brace, said first sheet affixed to a portion of said flexible portion, said port passing through said flexible portion, said port attached to said first sheet with a sealed connection, and said port configured to receive the medication for introduction into a cavity defined by said first sheet and said second sheet.

8. The apparatus of claim 1 further including a third sheet that covers entirely said second sheet, said third sheet being impermeable to the medication, and said third sheet being removable from said second sheet, thereby exposing said second sheet for delivery of the medication through said second sheet.

9. A medication delivery apparatus for dispensing a medication to a patient over a period of time, said apparatus comprising:
a bladder having a first sheet and a second sheet, said first sheet being impermeable to the medication, said second sheet being permeable to the medication, said first sheet and second sheet joined together at a peripheral edge to define a cavity; and
a port configured to receive the medication, said port being sealable after receiving the medication, and said port in fluid communication with said bladder.

10. The apparatus of claim 9 wherein said first sheet includes an opening, and said port positioned proximate to said opening.

11. The apparatus of claim 9 wherein said port extends longitudinally from a peripheral edge of said medication delivery device, said port attached to said peripheral edge at a neck, said port having a distal end with an opening into a passageway into said bladder.

12. The apparatus of claim 9 further including a third sheet that covers entirely said second sheet (104), said third sheet being impermeable to the medication, and said third sheet being removable from said second sheet, thereby exposing said second sheet for delivery of the medication through said second sheet.

13. The apparatus of claim 9 wherein said first sheet includes an adhesive whereby said bladder is attachable to an inside surface of a brace.

14. The apparatus of claim 9 wherein said first sheet includes a weak adhesive whereby said bladder is releasably attachable from an inside surface of a brace.

15. The apparatus of claim 9 further including a brace, said brace configured to attach to the patient, and said brace having a patient-facing side, and said bladder affixed to said patient-facing side of said brace.

16. The apparatus of claim 15 wherein said first sheet is attached to said brace with a weak adhesive such that said medication delivery device is removable from said brace.

17. The apparatus of claim 15 wherein said bladder is integrated with said brace, said first sheet affixed to a portion of said brace, said port passing through said portion, said port attached to said first sheet with a sealed connection, and said port configured to receive the medication for introduction into said cavity defined by said first sheet and said second sheet.

18. A medication delivery apparatus for dispensing a medication to a patient over a period of time, said apparatus comprising:
a brace configured to attach to the patient, said brace having a patient-facing side; and
a medication delivery device having a distal sheet and a releasable sheet, said medication delivery device positioned on said patient-facing side of said brace, said distal sheet being a membrane that is permeable to the medication, said releasable sheet being impermeable to the medication, said releasable sheet being weakly secured to said distal sheet, and said releasable sheet being removable from said distal sheet thereby exposing said distal sheet for delivery of the medication through said distal sheet.

19. The apparatus of claim 18 wherein said medication delivery device includes a bladder sized and configured to fit in a recess in said patient-facing side of said brace, said bladder having a proximal sheet and said distal sheet, said proximal sheet being impermeable to the medication, said proximal sheet and distal sheet joined together at a peripheral edge to define a cavity; and a port configured to receive the medication, and said port being sealable after receiving the medication, and said port in fluid communication with said bladder.

20. The apparatus of claim 19 wherein said bladder is adhered in said recess with a weak adhesive, whereby said medication delivery device is removable from said brace.

21. The apparatus of claim 18 wherein said medication delivery device is integrated with said brace, said brace having a patient-facing side that is impermeable to the medication, said distal sheet secured to said patient-facing side with a sealed connection, said port passing through a flexible portion of said brace, and said port configured to receive the medication for introduction into a cavity defined by said flexible portion and said distal sheet.

22. The apparatus of claim 18 wherein said medication delivery device includes a plurality of vesicles disposed proximate said distal sheet.

23. The apparatus of claim 18 wherein said medication delivery device includes a plurality of vesicles disposed proximate said distal sheet, and said medication delivery device sized and configured to fit in a recess in said patient-facing side of said brace.
